(19) United States
(12) Patent Application Publication
(10) Pub. No.: US 2024/0122964 A1
(43) Pub. Date: Apr. 18, 2024

(54) COMPOSITIONS AND METHODS TO REDUCE INFILTRATION OF PATHOGENIC AND ANTHROPOGENIC ENVIRONMENTAL FACTORS INTO THE BRAIN

(71) Applicant: Shawn E. WALKER, Andover, MA (US)

(72) Inventor: Shawn E. WALKER, Andover, MA (US)

(21) Appl. No.: 18/541,398

(22) Filed: Dec. 15, 2023

Related U.S. Application Data
(63) Continuation of application No. PCT/US2022/044032, filed on Sep. 19, 2022.
(60) Provisional application No. 63/245,716, filed on Sep. 17, 2021.

(51) Int. Cl. A61K 31/722; A61K 9/00; A61K 31/727; A61P 31/14; A61P 39/00

(52) U.S. Cl. CPC A61K 31/722; A61K 9/0075; A61K 31/727; A61P 31/14; A61P 39/00

(57) ABSTRACT

The invention provides methods and compositions for blocking access to, and reducing infiltration into, the brain of a subject through the subject's olfactory nerves, olfactory support structures, or both, of one or more pathogenic or anthropogenic environmental factor by contacting the surface of the olfactory epithelium with a polyanionic polysaccharide-chitosan composition.

### CROSS-REFERENCE TO RELATED APPLICATIONS

This application is a continuation of PCT/US2022/044032, filed Sep. 19, 2022, which claims priority to, and the benefit of, U.S. Provisional Patent Application No. 63/245,716, filed Sep. 17, 2021, the contents of which are incorporated herein by reference for all purposes.

### STATEMENT OF FEDERAL FUNDING

Not applicable.

### PARTIES TO JOINT RESEARCH AGREEMENT

Not applicable.

### BACKGROUND OF THE INVENTION

The olfactory epithelium, or “OE,” and olfactory mucosa, or “OM,” is distinguished anatomically and physiologically from the nasal epithelium, nasal mucosa, respiratory epithelium and respiratory mucosa. The OE is directly accessible from the external environment and exposed to a variety of environmental insults. While the OE has several barriers to such insults, ranging from physical to enzymatic and immune defenses, these barriers deteriorate with age and exposure, rendering them more permeable to xenobiotics, pathogens, and toxins.

Perhaps due to its vulnerable location, the olfactory mucosa has several different defense mechanisms against pathogen and dysbiont entry and cellular damage by environmental exposure. (Dysbiosis is now suspected of playing a role in neurodegenerative disorders. See, e.g., Harrass, et al., Intl J Molecular Sci, 2021, 22:11207; doi.org/10.3390/ijms222011207.) Bowman's glands secrete the olfactory mucus, which contains immune factors, lysozymes, enzymes, antioxidants, and possibly xenobiotic-metabolizing enzymes capable of viral inactivation, detoxification, bacterial degradation, and destruction of pro-inflammatory molecules. The mucus layer in which cilia of olfactory receptor neurons (sometimes referred to herein as “ORNs”) are immersed provides electrical insulation to the neurons and catches particles and odorants in suspension in the air. Sustentacular cells maintain water and salt balance in the mucus and metabolize xenobiotics. Sustentacular cells may also phagocytose debris and dying cells. The dense network of tight junctions between sustentacular cells and olfactory receptor neurons, and between the serous cells of Bowman's glands, reduce or prevent infiltration of pathogens into the OE.

Damaged olfactory receptor neurons, unlike other neurons, are constantly replaced by neurons that differentiate from the globose cells that derive from the horizontal basal cells. Until these cells mature and emerge anew in the olfactory epithelium, a gap remains in the space occupied by the previous ORN, disrupting tight junction formation. See, e.g., Crowe, et al., Life Sci., 2018, 195:44-52. In case of damage to the OE or contamination, neutrophils and macrophages can invade the olfactory mucosa though the lamina propria. Ensheathing cells protect and electrically isolate the axons of olfactory receptor neurons. Nerve bundles are protected by fibroblasts enveloping them. The microglial population localized in nerve bundles and in external layers of the olfactory bulbs is believed to be in a constant state of alert and acts as “sentinels” for the microglia in the rest of the brain. These microglia serve as sensors and modulators of inflammation for the entire brain.

Among the external factors that deteriorate the OE are respiratory pathogens and external environmental insults that impair tight junctions between the epithelial cells of the nasal epithelium (“NE”) and the OE, increasing epithelial permeability to external substances. Also, the OE dramatically changes with age; the olfactory receptor nerves can undergo necrosis or cease to be regenerated, and the OE itself becomes thinner Nasal mucociliary clearance is also less effective in the elderly and in certain disease states. Thus, as a consequence of infection, environmental stresses, and aging, the OE becomes more vulnerable to external threats.

External agents (bacteria, viruses, fungi, toxins, airborne pollutants, prions, micro- and nanoparticles) can trigger disease via the OE. These agents gain access to the brain through the OE to the olfactory bulbs (“OBs”) and spread along its associated connections, or they trigger protein misfolding locally in the OE and OBs that, in turn, leads to prion-like propagation of protein aggregates like amyloid, tau and synuclein-a via olfactory pathways. The OBs are one of the few segments of the central nervous system that is not protected by the blood brain barrier. The olfactory region is effectively a “neuroimmune” zone due to its anatomically vulnerable location between the outside world and the central nervous system.

Olfactory receptor neurons have dendrites that project into the nasal cavity, and axons that extend through the cribiform plate to the olfactory plate. See, e.g., Van Riel et al., J Pathol., 2015, 235(2): 277-87. doi: 10.1002/path.4461. van Riel et al. list the following as viruses that can use the olfactory nerve to access the central nervous system: influenza A virus, herpesviruses, poliovirus, paramyxoviruses, vesicular stomatitis virus, rabies virus, parainfluenza virus, adenoviruses, Japanese encephalitis virus, West Nile virus, chikungunya virus, La Crosse virus, mouse hepatitis virus, and bunyaviruses. A variety of mechanisms have been posed as to how such agents are transported along the ORNs, including travel via axonal transport, within the olfactory ensheathing cells, or within the perineural space, as well passing through the cribriform plate to access the subarachnoid space. Once within the OBs, viruses can migrate to other brain regions. Other pathogens, such as bacteria, can access the brain through the OE. Staphylococcus aureus has been shown to bypass compromised OE and enter the brain within six hours of challenge.

Exposure to environmental toxins, some of which are airborne and of sizes or in forms that can gain access to the olfactory system, has been associated with increased neurodegenerative disease risk. Airborne pollutants, environmental agents and xenobiotics can penetrate the central nervous system through the olfactory route, producing effects such as the inflammation, oxidative stress, increased apoptosis and dysfunction of mitochondria and proteasomes found in Parkinson's Disease (“PD”). All of these effects could affect neuronal function and protein aggregation. The risk of developing Alzheimer's Disease (“AD”) is increased by exposure to pesticides, viruses, pollutants and metals. Amyotrophic lateral sclerosis (“ALS”) is also associated with exposure to pesticides, metals, solvents, toxins, viruses, and possibly to pollutants. The prion disease Creutzfeldt-Jakob Disease, or “CJD,” is believed to be caused by a misfolded protein referred to as PrP^{Sc}. Animal models of PrP^{Sc} spread show that the nasal route is more efficient for prion infection than the gastric route and that in infected hamsters, OE damage favors the release of prions in the nose, possibly increasing transmission. Studies have shown that occupational exposure to manganese can result in Parkinsonism. Chronic high- (>1 mg/m³) and low-levels (0.5-1.0 mg/m³) of Mn inhalation exposures in the workplace have been reported to result in Mn accumulation in the brain and cause Mn-induced parkinsonism and subtle subclinical changes in the general population respectively. The concerns that chronic low-level Mn inhalation exposure may be associated with subtle, subclinical neurological changes have led to the development of pharmacokinetic data sets and physiologically based pharmacokinetic models (PBPK) in adult monkeys and rats, PBPK models of gestation and lactation in rat, and a PBPK model in humans [to predict inhalation exposure conditions that result in increased brain Mn levels. The PBPK model structure is comprised of compartments for the liver, lung, nasal cavity, bone, blood, cerebellum, olfactory bulbs, globus pallidus, and pituitary gland with the remaining body tissues combined into a single compartment, to name a few. The PBPK model simulates concurrent exposure to dietary and inhaled Mn and also simulates ⁵⁴Mn tracer kinetics from oral and inhalation exposure by intraperitoneal (ip), intravenous (iv), and subcutaneous (sc) administration. Kwakye, et al., “Manganese-Induced Parkinsonism and Parkinson's Disease: Shared and Distinguishable Features,” Int. J. Environ. Res. Public Health 2015, 12, 7519-7540. Studies have shown that Neurodegenerative diseases (NDs) such as Alzheimer's and Parkinson's disease are fatal neurological diseases that can be of idiopathic, genetic, or even infectious origin, as in the case of transmissible spongiform encephalopathies. The etiological factors that lead to neurodegeneration remain unknown but likely involve a combination of aging, genetic risk factors, and environmental stressors. Accumulating evidence hints at an association of viruses with neurodegenerative disorders and suggests that virus-induced neuroinflammation and perturbation of neuronal protein quality control can be involved in the early steps of disease development. Leblanc P, Vorberg I M (2022) Viruses in neurodegenerative diseases: More than just suspects in crimes. PLoS Pathog 18(8): e1010670.

In addition to neurodegenerative diseases, exposure to environmental toxins such as air pollutants and respiratory infections has also been associated with psychiatric disorders such as schizophrenia and depression. Hasegawa Y, et al. (2022) Olfactory impairment in psychiatric disorders: Does nasal inflammation impact disease psychophysiology? Trans Pysch 12(314), doi: 10.1038/s41398-022-02081y. Olfactory performance is often impaired in patients with schizophrenia or depression, which is associated with further social dysfunction and negative symptoms. The pathophysiologic role of airborne pollutants and environmental toxins on the development of psychiatric disorders is not fully understood, but thought to be due at least in part to inflammation.

Many xenobiotics and particles that can penetrate the brain through the OE are known to trigger reactive oxygen species (“ROS”) production and inflammatory reactions. As an interface between the external environment and the brain, the OE and OBs are particularly subject to inflammatory reactions. The OBs and olfactory nerves host dense populations of microglia that act to prevent particles and pathogens from penetrating the brain. The microglia of the OBs show more rapid and higher levels of activation for longer periods (lasting for months) relative to microglia in other regions of the brain. OB microglia can also be activated by injury or infections occurring in distant sites of the brain and might play a role of sentinel for the whole brain (Lalancette-Hebert et al., 2009; 132(4):940-54, DOI:10.1093/brain/awn345; Smithson and Kawaja, J Neurosci Res. 2010;88(4):858-65. doi: 10.1002/jnr.22254.2010). Activated microglia release pro-inflammatory cytokines and ROS that can damage cells. These proinflammatory cytokines and ROS can disrupt the blood-brain barrier, thus facilitating the penetration of xenobiotics and particles into the brain.

Hence, a feed-forward loop of inflammation, involving a complex interplay of ROS production and protein aggregation, could play an important role in the pathogenesis of neurodegenerative and neurological diseases. Anterior olfactory regions could be particularly vulnerable due to the presence of highly sensitive microglia and a blood-brain barrier that is easily compromised.

Due to the specific environment within the central nervous system, it is difficult for systemically administered treatments to either reach the appropriate environment, or to achieve the concentration necessary to impact disease initiation and progression. Administration within the central nervous system also has challenges, such as directing treatment to the appropriate place in the central nervous system and penetrating the blood brain barrier and mucosal layer of the upper respiratory and olfactory epithelium. Molecule size, toxicity, pH, and muco-ciliary clearance create further challenges for effective olfactory mucosal delivery.

Delivery of therapeutics to the brain through the olfactory mucosa has been recognized as a possible method of avoid some of the challenges of systemic drug administration, including first-pass clearance and the need to provide high systemic exposure to an agent to achieve a therapeutic level of the agent within the brain. Unfortunately, direct nose-to-brain drug delivery has been limited by “the extremely low delivery efficiency (<1%) of conventional devices to the olfactory region” due to “the complexity of the nasal structure that traps particles before reaching the olfactory region.” Xi, et al., Int J Nanomedicine, 2015; 10:1211-1222. doi: 10.2147/IJN.S77520.

Su, et al. (Pharmaceutics 2020, 12(10):907; doi.org/10.3390/pharmaceutics12100907) reported the use of a sodium glycerophosphate/chitosan hydrogel doped with PEG-PLA nanoparticles bearing a microRNA for intranasal delivery of the microRNA in a rodent model.

There remains a need in the art for a non-toxic, prophylactic treatment that can reduce exposure to environmental factors associated with increased risk of neurodegenerative and psychiatric diseases or transmission of neurotropic infectious disease of various etiologies. There is a further need to block access of undesired environmental factors to the brain through the olfactory epithelium. Surprisingly, the present invention fulfills these and other needs.

### SUMMARY OF THE INVENTION

In some embodiments, the invention provides methods of blocking access of at least one pathogenic and anthropogenic environmental factor to olfactory epithelium having a proximal side, which proximal side faces an interior surface of a nose of a subject, said method comprising contacting said proximal side of the olfactory epithelium with an effective amount of a chitosan complex or chitosan composition, thereby blocking access of the environmental factors to said olfactory epithelium, provided that the chitosan complex or chitosan composition does not comprise an effective amount of a therapeutic agent to be transported into the brain of the subject. In some embodiments, the chitosan is 75% or more deacetylated.

In some embodiments, the chitosan complex comprises a polyanionic polysaccharide complexed with said chitosan. In some embodiments, the chitosan and the polyanionic polysaccharide are present in said composition in a ratio from about 100:1 to 10:1. In some embodiments, the polyanionic polysaccharide is selected from the group consisting of heparin, dextran sulfate, heparan sulfate, and chondroitin sulfate. In some embodiments, the polyanionic polysaccharide is an anti-coagulant form of heparin. In some embodiments, the polyanionic polysaccharide is a non-anti-coagulant form of heparin. In some embodiments, the chitosan complex comprises sialic acid complexed on to said chitosan. In some embodiments, the chitosan complex comprises sialic acid complexed with the chitosan. In some embodiments, the chitosan complex consists essentially of chitosan complexed with a polyanionic polysaccharide, chitosan complexed with a sialic acid, or chitosan complexed with both a polyanionic polysaccharide and a sialic acid.

In some embodiments, if the chitosan complex is provided as a hydrogel, the chitosan complex does not comprise nanoparticles. In some embodiments, the environmental factor is a pathogen. In some embodiments, the pathogen is a bacterium, a fungus, a parasite, a prion, or a virus. In some embodiments, the virus is a coronavirus. In some embodiments, the coronavirus is SARS-CoV-2. In some embodiments, the environmental factor is a pesticide. In some embodiments, the environmental factor is particulate matter. In some embodiments, the particulate matter is Particulate Matter (PM)_{2.5}. Exposure to PM induces prominent cognitive decline in humans and laboratory animals due to structural changes and atrophy in the gray and white matter. The hippocampus and glutamatergic neurons played crucial roles in the learning and memory; while PM exposure affected these neurons structurally and functionally. Glutamatergic neurons are susceptible to the insults of PM because oxidative stress induced by PM aggravates the glutamatergic excitotoxicity in neurons. PM2.5 dose—dependently decreased the cell viability of primary human neurons and increased the levels of cleaved caspase-3. A 48-h treatment with nano scaled PM inhibited the neurite outgrowth in a primary culture of rat hippocampal neurons. Evidence from numerous epidemiological and experimental studies have indicated PM as a risk factor for neurodegenerative diseases and psychiatric disorders, especially Alzheimer's disease (AD), Parkinson's disease (PD), schizophrenia, and depression. AD is characterized by progressive cognitive impairment, accompanied by neuronal death, neuroinflammation, and the accumulation of two neuropathological markers, i.e., senile plaque composed of amyloid-β (Aβ) peptides and neurofibrillary tangles accumulated from hyperphosphorylated tau. Patients with PD symptomatically manifest motor deficits due to the loss of dopaminergic neurons. PD is pathologically characterized by Lewy bodies formed by a-synuclein aggregation.

In some embodiments, the particulate matter is comprised of traffic-related particles of tire wear, brake wear, exhaust, e.g., gasoline, diesel, or other fuel exhaust.

In some embodiments, the chitosan complex or chitosan composition is atomized In some embodiments, the chitosan complex or chitosan composition is applied as a dry powder. In some embodiments, the chitosan complex or chitosan composition is applied as a spray. In some embodiments, the chitosan complex or chitosan composition is administered via a device configured to apply substances to olfactory epithelium. In some embodiments, the chitosan complex or chitosan composition is administered for at least one week. In some embodiments, the chitosan complex or chitosan composition is administered for at least two weeks. In some embodiments, the chitosan complex or chitosan composition is administered for at least three weeks. In some embodiments, the chitosan complex or chitosan composition is administered for at least one month. In some embodiments, the contacting is with an effective amount of said chitosan complex or chitosan composition.

In some embodiments, the invention provides methods of reducing infiltration into the brain of a subject through said subject's olfactory nerves, olfactory support structures, or both, of at least one pathogenic or anthropogenic environmental factor. The methods comprise contacting a proximal side of said olfactory nerves, said olfactory support structures, or both, with an effective amount of a chitosan complex or chitosan composition, thereby reducing the infiltration of at least one pathogenic or anthropogenic environmental factor into the olfactory nerves, the olfactory support structures, or both, provided the composition comprising chitosan does not comprise an effective amount of a therapeutic agent to be transported into said brain of said subject. In some embodiments, the infiltration of said environmental factor is reduced by at least 5%. In some embodiments, the infiltration of said environmental factor is reduced by at least 10%. In some embodiments, the infiltration of said environmental factor is reduced by at least 20%. In some embodiments, the infiltration of said environmental factor is reduced by at least 25%. In some embodiments, the chitosan is 75% or more deacetylated.

In some embodiments, the chitosan complex comprises a polyanionic polysaccharide complexed on said chitosan. In some embodiments, the chitosan and said polysaccharide are present in said composition in a ratio from about 100:1 to 10:1. In some embodiments, the polysaccharide is selected from the group consisting of heparin, dextran sulfate, heparan sulfate, and chondroitin sulfate. In some embodiments, the polyanionic polysaccharide is a non-anticoagulant form of heparin. In some embodiments, the polyanionic polysaccharide is an anti-coagulant form of heparin. In some embodiments, the chitosan complex further comprises sialic acid immobilized to chitosan. In some embodiments, the chitosan complex consists essentially of chitosan complexed with a polyanionic polysaccharide, of chitosan complexed with a sialic acid, or of chitosan complexed with both a polyanionic polysaccharide and a sialic acid.

In some embodiments, the chitosan complex is provided as a hydrogel, it does not comprise nanoparticles. In some embodiments, the contacting is with an effective amount of said chitosan complex or chitosan composition.

In some embodiments, the environmental factor is a pathogen. In some embodiments, the pathogen is a bacterium, a fungus, a parasite, a prion, or a virus. In some embodiments, the virus is a coronavirus. In some embodiments, the coronavirus is SARS-CoV-2. In some embodiments, the environmental factor is a pesticide. In some embodiments, the environmental factor is a particulate mix from an industrial or work operation. In some embodiments, the environmental factor is a metal atom or ion. In some embodiments, the chitosan complex or chitosan composition is atomized In some embodiments, the chitosan complex or chitosan composition is applied as a dry powder. In some embodiments, the chitosan complex or chitosan composition is applied as a spray. In some embodiments, the chitosan complex or chitosan composition is administered via a device configured to apply substances to olfactory epithelium. In some embodiments, the chitosan complex or chitosan composition is administered for at least two weeks. In some embodiments, the chitosan complex or chitosan composition is administered for at least one month.

In some embodiments, the invention provides a method of reducing infiltration into the brain of a subject through said subject's olfactory nerves, olfactory support structures, or both, of a metal atom or ion, said method comprising contacting a proximal side of said olfactory nerves, said olfactory support structures, or both, with an effective amount of a composition comprising alpha-chitosan complexed with a polyanionic polysaccharide, thereby reducing said infiltration of said metal atom or ion into said olfactory nerves, said olfactory support structures, or both, wherein said composition does not comprise an effective amount of a therapeutic agent to be transported into said brain of said subject, and wherein said composition has a pH from about 4 to about 6.

In some embodiments, the invention provides a method of reducing infiltration into the brain of a subject through said subject's olfactory nerves, olfactory support structures, or both, of a particulate matter, said method comprising contacting a proximal side of said olfactory nerves, said olfactory support structures, or both, with an effective amount of a composition comprising alpha-chitosan complexed with a polyanionic polysaccharide, thereby reducing said infiltration of said particulate matter into said olfactory nerves, said olfactory support structures, or both, wherein said composition does not comprise an effective amount of a therapeutic agent to be transported into said brain of said subject, and wherein said composition has a pH from about 4 to about 6. In some embodiments, the particulate matter is PM_{2.5}.

Thus, the present disclosure provides compositions and methods for inhibiting factors that contribute to development of NDs by blocking access to the brain of such factors.

### BRIEF DESCRIPTION OF THE DRAWINGS

FIG. 1 is a graph showing the results of a study whether heparin/chitosan compositions inhibit binding of SARS-CoV-2 spike protein to susceptible cells. Human A549 cells (a cell line of adenocarcinomic human alveolar basal epithelial cells) were either left untreated, were contacted with an enzyme to degrade heparan sulfate on the surface of the cells, or were contacted with a heparin/chitosan composition applied at any of four different concentrations shown in the legend. The groups of cells were then incubated with recombinant SARS-CoV-2 S1 and S2 viral spike protein. Binding of the viral spike protein to the cells was assessed using a fluorescent antibody and analyzed by flow cytometry. “UT”=untreated. “HSase”=cells treated with an enzyme to degrade heparan sulfate on the surface of the cells. The results show that the binding of SARS-CoV-2 spike protein to the cells was inhibited by the heparin/chitosan composition in a dose-dependent manner.

FIG. 2 provides results of chitosan alone or heparin/chitosan compositions binding to the metal Mn.

FIG. 3 shows the results of a study of preventing infiltration of Mn into the brains of living rats using heparin/chitosan compositions at 4 hours. The ratios of [⁵²Mn] in whole brain, or olfactory bulbs, to both total injected dose and activity measured from the residual head following brain/olfactory bulbs removal as an estimate of signal retained within the nasal cavity. From left to right, the bars for each group represent “Brain to Injected Dose,” “Brain to Residual Head,” “Olfactory Bulbs to Injected Dose,” and “Olfactory Bulbs to Residual Head.”

FIG. 4 shows ex vivo percent injected dose (%ID) in tissue and excreta of [⁵²Mn] at 24 hours post-tracer administration. From left to right, the bars for each group represent “Carcass,” “Excreta,” “Olfactory Bulb,” “Residual Head,” and “Whole Brain.”

### DETAILED DESCRIPTION

As set forth in the Background, the olfactory region is an anatomically vulnerable region between the environment outside the body and the central nervous system. As described, for example, by Smith and Bhatnagar, Handb Clin Neurol, 2019, 164:17-28, olfactory neurons extend long ciliary processes into the olfactory mucus, with odorant receptors disposed on the cilia. The axons of the olfactory neurons extend into the brain and synapse within the sensory bulbs. Some metals (e.g., Mn, Ni, Zn) can cross synapses in the olfactory bulbs and migrate via secondary olfactory neurons to distant nuclei of the brain. After nasal instillation of a metal-containing solution, transport of the metal via olfactory axons can occur rapidly, within hours or a few days (e.g., Mn), or slowly over days or weeks (e.g., Ni). The olfactory bulbs tend to accumulate certain metals (e.g., Al, Bi, Cu, Mn, Zn) with greater avidity than other regions of the brain. Such heavy metals can have detrimental health effects. The present disclosure provides methods of inhibiting or reducing infiltration into the brain of a subject through said subject's olfactory nerves, olfactory support structures, or both, of one or more metal atom or ion. For example, the metal may be Al, Bi, Cu, Mn, Zn, Ni, Pb, Mg, Fe, Cd, Cu, Hg, As, and the like.

Recent studies have provided evidence that a number of pathological conditions are caused by, or aggravated by, infiltration of pathogenic and anthropogenic environmental factors into the brain. Such environmental factors include bacteria, viruses, fungi, toxins, prions, and airborne pollutants and particulates such as pesticides, heavy metals, and particulates, e.g., from diesel, gasoline, or other traffic-related particulates, mineral dust, industrial emissions, or generated through chemical reactions of other airborne pollutants, such as nitrogen oxides, printer inks, heavy metals, and organic compounds. See You, R., Ho, Y S. & Chang, R.C C. The pathogenic effects of particulate matter on neurodegeneration: a review. J Biomed Sci 29, 15 (2022). Further, recent studies have indicated that these toxic environmental factors have gained access to the brain by transport along the axons of olfactory neurons from the nose to the olfactory bulbs, by infiltration following disruption of junctions of cells in the olfactory region, by uptake by microglia or other accessory cells, or by transport in or along periolfactory spaces.

Respirators and high-filtration N95 masks can reduce access of such factors to the nose and, thus, reduce their access to the olfactory neurons. Such external barriers, however, suffer from a number of limitations on their use. They must be very tightly fitted, which makes them uncomfortable. They are inconvenient to sleep in and are often ineffective for use during sleep, as the user can dislodge them while asleep. Further, they cannot be worn while eating or drinking, requiring the user to either move to an area with lower levels of the environmental factors, or to undergo exposure to the factors while drinking or eating. Finally, most external barriers and surgical masks do not protect the wearer from the most problematic environmental factors due to their inability to filter out the smallest particle sizes.

The ability to access the brain has led a number of researchers to investigate introducing drugs into the brain through the olfactory region. A number of such efforts are reviewed, for example, in Erdo et al., Brain Research Bulletin, 2018, 143:155-170.

The present invention turns these efforts on their heads. The present invention stems from the surprising realization that, rather than using the “nose-to-brain” route of administration to get therapeutic agents into the brain, selected chitosan complexes and chitosan compositions can instead be applied to the olfactory epithelium to reduce or to prevent the infiltration of environmental factors into the brain by providing a physical barrier to their entry. Further, it is believed that the chitosan complexes and chitosan compositions capture environmental factors and that, as the chitosan complexes and compositions degrade, divert the environmental factors to the gastric tract, resulting in their elimination rather than allowing them access to the olfactory epithelium and, through the epithelium, to the brain.

Without wishing to be bound by theory, it is believed that toxic or otherwise unwanted environmental factors bind to the chitosan or to one or more polyanionic polysaccharides or, in embodiments in which it is present, to the sialic acid, thereby preventing them from reaching the olfactory neurons and olfactory support structures. It is further believed that, once bound, the environmental factors trapped on the chitosan complex or composition are gradually released into the olfactory mucus as the complex or composition is degraded and individual molecules or small groups of chitosan, along with adsorbed environmental factors are swept along with olfactory mucus and respiratory mucus into the nasopharynx for expectoration or are swallowed into the digestive tract for excretion. For those environmental factors that are pathogens (e.g., bacteria, viruses, fungi, parasites and prions), it is believed that holding the pathogen on the chitosan complex or composition until the composition degrades also allows time for the pathogen to die or otherwise be neutralized before being swept into the gastric tract, further reducing the pathogen's potential for causing harm to the subject. See, e.g., Mycroft-West et al. Thromb Haemost 2020; 120(12):1700-1715. doi.org/10.1055/s-0040-1721319. In some embodiments, the practitioner may desire to reduce the exposure of a subject to an airborne pathogen known or suspected to bind to heparin or use heparan sulfate as an adhesion site receptor in the subject's environment for a period of time. For example, clinicians treating infected patients or incarcerated persons to reduce transmission of Covid-19 may choose to apply either a chitosan complex or a composition that would have higher polyanionic polysaccharide concentrations, due to their ability to adsorb and neutralize pathogens.

Prions are misfolded proteins, rather than living organisms, but are able to self-propagate. For convenience, they are included in the methods discussed herein as a “pathogen” whose infiltration into the brain can be reduced or blocked using the inventive methods. While these misfolded proteins are considered to be non-living, they cannot “die” before being swept into the digestive tract. It is expected, however, that prions will be immobilized on chitosan compositions applied to the OE, will be sequestered from the brain of subjects in which the compositions have been applied, and will eventually be swept into the digestive tract for disposal. Embodiments of the inventive methods are expected to reduce or to prevent prions from accessing the olfactory epithelium and, through the OE, from accessing the olfactory bulbs.

The blocking of prions can also occur in the reverse direction. Prions have been found in the olfactory mucosa of humans afflicted with the prion disease Creutzfeldt-Jakob disease (“CJD,” see, Zanusso et al., N Engl J Med 2003; 348:711-719. DOI: 10.1056/NEJMoa022043) and in the nasal secretions of non-human animals with at least one prion disease. The nasal secretions are believed to be a source of transmission of these dangerous infective agents. See, e.g., Bessen et al., PLoS Pathog 2010, 6(4): e1000837. doi.org/10.1371/journal.ppat.1000837; Bessen et al., J Virol. 2012, 86(3): 1777-1788. doi: 10.1128/JVI.06626-11. Chitosan compositions can be administered to the OE of individuals with CJD or another prion disease to trap prions on the chitosan and reduce the titer of prions into the individuals' nasal mucus and subsequent discharge, thereby reducing the exposure to prions of those providing care to the afflicted individuals.

In some embodiments, the inventive methods comprise administering an aerosolized complex of chitosan and a polyanionic polysaccharide immobilized thereto to the olfactory epithelium of the subject. The positive charge of the chitosan allows the complex to adhere to the negatively-charged surface of the olfactory epithelial cells, and olfactory mucosa, forming a barrier layer that serves as a physical blockade keeping the undesired environmental factors from reaching the surface of the olfactory epithelium to which the chitosan complex has adhered. Without wishing to be bound by theory, it is believed that the architecture of the polyanionic polysaccharide serves as a decoy receptor for pathogens that would normally bind cells, effectively neutralizing them before they can cause infection.

In some embodiments, the toxic environmental factor blocked by the chitosan complex or composition is a metal atom or ion. For example, Mn and Pb are known to cause long-lasting neurotoxicities (see Neal A P, Guikarte T R. Mechanisms of lead and manganese neurotoxicity. Toxicol Res (Camb). 2013 March 1:2(2):99-114. doi: 10.1039/C2TX20064C). A person can be exposed to metals through jobs like mining or electronics recycling, or through environmental pollution of gas combustion or industrial emissions. A practitioner may desire to reduce uptake of toxic metals in high-risk individuals. According to these embodiments, the present disclosure includes methods of applying a chitosan complex or composition that binds metal atoms or ions. In the experiments performed herein, manganese (Mn) is used as a representative of heavy metals. Studies have shown a similar uptake between manganese and various heavy metals. Mehra and Thakur “Relationship between lead, cadmium, zinc, manganese and iron in hair of environmentally exposed subjects,” Arabian Journal of Chemistry, Vol. 9, Suppl. 2, 2016, S1214-S1217. Thus, the results obtained herein apply to other heavy metals. Studies have shown that such metals can directly access the brain via the olfactory area. Sunderman, “Nasal Toxicity, carcinogenicity, and olfactory uptake of metals,” Annals of Clinical & Laboratory Science, Vol. 31, no. 1, 3-24, Winter 2001.

In some situations, a practitioner may wish to reduce a subject's exposure to environmental factors that are believed to contribute to neurodegenerative or psychiatric disorders. For example, persons with a variation of the apolipoprotein E gene called APOE4 have one of the most significant genetic risk factors for developing Alzheimer's Disease, or “AD”. It is estimated that about 25% of the population has one allele (copy) of APOE4 and that 2-3% carry two copies. A practitioner might determine that subjects with one copy of APOE4, and especially those having two copies, would benefit from reducing the risk that the subject might be pushed towards AD by exposure to environmental factors. In this situation, the practitioner would likely recommend application on an ongoing basis of a chitosan complex or composition that binds a wide range of environmental factors to reduce exposure to those factors and, current evidence suggests, thereby prevent or delay onset of AD or slow its progression. Administration of the chitosan complexes or compositions in these embodiments will be for months or years at a time, in the hope of preventing or slowing progression of the neurodegenerative condition.

In some embodiments, a practitioner may desire to reduce exposure of a subject to a particular environmental factor expected to be in the subject's environment for a limited period of time. For example, persons applying pesticides to fields of a farm over a day or a period of days may only need to reduce their exposure to that one pesticide and only for the day or days on which the pesticide will be applied to the fields. For this use, the practitioner can choose to apply either a chitosan complex or composition that binds the pesticide to be applied, but not a broad range of other environmental factors, or a chitosan complex or composition that binds a broad range of environmental factors that includes the pesticide to be applied. Similarly, truckers waiting for hours among dozens of trucks picking up cargo containers at a seaport may face high levels of pollution from diesel engine exhaust. In this situation, the practitioner can choose to apply either a chitosan complex or composition that binds particulates from diesel engine exhaust, but not a broad range of other environmental factors, or a chitosan complex or composition that binds a broad range of environmental factors that includes particulates from diesel engine exhaust. In some other embodiments, such as first responders at disaster sites, service members exposed to burn pits, or firefighters responding to wildfires, the subjects are or will be exposed to a spectrum of airborne toxins for days, and sometimes weeks, on end. In these circumstances, practitioners would choose to apply a chitosan complex or composition that binds a wide range of environmental factors for the duration of the user's service at these locations. Without wishing to be bound by theory, it is believed that these pollutant (non-living) environmental factors can be captured effectively by compositions of chitosan alone, or by chitosan/heparin complexes in which the chitosan component is present at the higher end of the ratios discussed below, while pathogens will bind more effectively to the heparin component of chitosan/heparin complexes and will be captured more effectively if the ratio of heparin to chitosan is higher than for compositions intended to target only environmental pollutants such as heavy metals. Chitosan/heparin complexes or compositions intended to capture both environmental pollutants and pathogens may typically use a more balanced ratio of chitosan to heparin.

The Examples section sets forth assays by which a practitioner can readily test chitosan compositions of chitosan alone, chitosan complexed with one or more sialic acids, chitosan complexed with a particular polyanionic polysaccharide, chitosan complexed with a combination of particular polyanionic polysaccharides, or chitosan complexed with both one or polyanionic polysaccharides and one or more sialic acid, to see which chitosan complex or composition binds any particular environmental factor, or any particular group or groups of environmental factors, whose infiltration into the brain of a subject the practitioner wishes to reduce.

Chitosan complexed with non-coagulant forms of heparin have previously been suggested for administration to the lungs or respiratory tract to treat respiratory inflammation. See, Shum et al., U.S. Patent Application Publication 2011/0212181 A1 (hereafter, “Shum,” incorporated herein by reference). Shum teaches that the compositions can be introduced into the lungs by inhalation of dry powder aerosols or in solutions that are nebulized for inhalation. Shum also states that mucosal formulations can be used to enhance “delivery through the nasal mucosa.” As the purpose for which Shum teaches administering its compositions is treating respiratory inflammation, it presumably is referring to systemic treatment via the nasal mucosa of the upper respiratory tract. Ahmed, U.S. Pat. No. 6,193,957 (hereafter, “Ahmed,” incorporated herein by reference), teaches that intrabronchial administration of what it terms “ultra-low molecular weight” heparin (heparins or other sulfated polysaccharides with an average molecular weight of 1,000-3,000 daltons) reduces airway hyperresponsiveness.

It is believed that persons following the teachings of either Shum or Ahmed would not practice the methods of the present invention, either deliberately or inadvertently. First, both Shum and Ahmed introduce their compositions to the subjects primarily through inhalation through the mouth. This is understandable, as both are directed to treatment of lung diseases and any of their formulations that reached the olfactory epithelium would be unavailable for the purpose for which the formulations were being administered. Second, neither Shum nor Ahmed teaches administration of their respective formulations to the olfactory epithelium. Again, this is not surprising, given the conditions which the respective investigators were trying to treat. Third, while it is relatively easy to deliver agents to the olfactory epithelium of rodents due to the configuration of their anatomy, as explained below, it is difficult in humans without using devices specially configured for the purpose, or without having the recipient assume a position, such as the Kaiteki position or the Mygind position, developed to allow the recipient to provide compositions to the olfactory cleft. See, e.g., Milk, et al., Clin. Otolaryngology, 2021, 46(2):406-411.

Air entering the nose passes through the nasal valve and over three bilateral structures, called conchae, protrusions which are also called the “turbinate bones.” As the name “turbinate” implies, the conchae cause the incoming flow of air to become turbulent.

The resulting swirl of air causes larger particles in the air flow to contact mucus lining the sides of the nose. The rear two thirds of the nasal cavity is lined with pseudostratified columnar ciliated epithelium. The cilia of the ciliated epithelial cells (see, e.g, Martinez-Giron, Acta Biomed, 2020, 91(1):146-147; doi: 10.23750/ abm.v91i1.8924) direct mucus, including mucus that has captured particles and pathogens from the nasal passages, to the stomach, where the captured particles and pathogens encounter stomach acid and digestive enzymes. Further, the olfactory slit has a small cross-sectional area relative to the lower nasal airway that allows a relatively small air flow. See, Kelly et al., J Appl Physiol, 2000, 89:323-337. As observed by Kelly et al., the “low-flow characteristic seems to be a defense mechanism that prevents particles whose trajectories are heavily dependent on flow patterns from being convected to and deposited on the sensitive olfactory nerve fibers, while allowing vapors to diffuse to that region for olfaction.” Kelly et al., at page 332. Based on these factors, it is believed that compositions administered following Shum or Ahmed would not result in deposition of the compositions on the olfactory epithelium or, if any such deposition happened to occur would not do so in amounts effective to reduce uptake of environmental factors by any appreciable degree, such as 5%, 10%, 15%, or 20%.

For convenience of reference, the surface of the olfactory epithelium facing into the nasal passages, and which therefore can be exposed to contact with environmental factors such as pollutants or pathogens is sometimes referred to herein as the “proximal surface” or the “proximal side,” as it is proximal to the external world, as opposed to the side of the epithelium facing into the internal structures of the brain. Finally, the proximal surface (or side) of the cells of the olfactory epithelium, including the olfactory nerves and support structures, are typically protected by a layer of mucus. It is understood that application of the chitosan compositions and chitosan complexes discussed herein to the proximal surface of the cells of the olfactory epithelium may result in the compositions or complexes being in contact with the mucus overlaying the cells instead of being in contact with the surface of the cells themselves, may be in contact with the surface of the cells themselves, or both. Without wishing to be bound by theory, it is believed that coating either the mucus overlaying the cells of the OE, or the cells of the OE themselves, with the chitosan compositions or chitosan complexes discussed herein, are both effective in blocking access of environmental factors to the brain, and in reducing infiltration of such factors into the brain. As used herein, “contacting a proximal side of olfactory epithelium with an effective amount of a” substance, such as a chitosan complex or a chitosan composition, means contacting the complexes or compositions to the proximal side of cells of the OE, to mucus overlaying and in contact with those cells, or to both.

### Chitosan Complexes and Compositions

As used herein, “chitosan” refers to alpha-chitosan, the abundant form of chitosan found in arthropods, algae, fungi, yeast, the shells of shrimp, crabs, lobsters, annelids, insects and protozoa. Alpha-chitosan has distinct structural and functional characteristics from squid pen-derived beta-chitosan, the latter of which is excluded from the embodiments herein. In some aspects, the composition of the present disclosure does not include beta-chitosan. In some aspects, the composition of the present disclosure is devoid of beta-chitosan.

As used herein, a “chitosan complex” or “chitosan composition” for use in embodiments of the inventive methods comprises alpha-chitosan and a polyanionic polysaccharide ionically complexed thereto, or comprises a combination of alpha-chitosan with a polyanionic polysaccharide ionically complexed thereto and with a sialic acid ionically complexed thereto. In some embodiments, the chitosan complex or composition consists essentially of chitosan and a polyanionic polysaccharide ionically complexed thereto, or consists essentially of a combination of chitosan with a polyanionic polysaccharide ionically complexed thereto and with a sialic acid ionically complexed thereto. The chitosan complex or composition may be a hydrogel, but preferably does not contain nanoparticles. In preferred embodiments, the complex or composition does not comprise an effective amount of a therapeutic agent intended by the practitioner to be introduced into the nasal mucosa or into the brain of the subject to whom the complex or composition is being administered.

As used herein, the transitional phrase “comprising” means “including,” the transitional phrase “consisting essentially of” limits the scope of a claim reciting it to the specified materials or steps and those that do not materially affect the basic and novel characteristics of the claimed invention, and the transitional phrase “consisting of” excludes any element, step, or ingredient not specified, unless the additional element, step, or ingredient is unrelated to the claimed invention, such as a buffer or excipient not expected to have any therapeutic effect or effect on the ability of the chitosan complex or composition to capture and sequester toxic environmental factors from the subject's OE. In preferred embodiments, the chitosan compositions do not contain nanosilver or plant essential oils.

Chitosan is a linear polysaccharide composed of randomly distributed β-(1→4)-linked D-glucosamine (deacetylated unit) and N-acetyl-D-glucosamine (acetylated unit). It is biocompatible, biodegradable, mucoadhesive, and exhibits favorable physiochemical properties (Lee, et al., Respri. Res., 2006, 7(112):1-10). Methods for producing chitosan microspheres by membrane emulsification are described in, for example, Wang et al., J. Control Release, 2005, 106(1-2):62-75. Microspheres or beads of uniform size can be prepared by utilizing membranes of different pore size. U.S. Patent Application Publication 2008/0202513 describes making dry powders comprising chitosan. For clarity, it is noted that, while chitosan is itself a polysaccharide, it is referred to in this disclosure as “chitosan” to avoid confusion with the polyanionic polysaccharides complexed with chitosan. References to “polysaccharide” other than in this paragraph (which describes chitosan) refer to polyanionic polysaccharides, not to chitosan.

Alpha-chitosan is manufactured by N-deacetylation of chitin, a polymer forming the shell of various insects, algae, fungi, and shellfish. By controlling the N-deacetylation reaction of chitin, chitosans of varying degree of N-acetylation can be made. In some preferred embodiments, an aerosolized composition of the present disclosure comprises chitosan that is preferably 95% or more deacetylated. In some embodiments, an aerosolized composition of the present disclosure comprises chitosan that is ≥75%-≤95% or more deacetylated. In some embodiments, an aerosolized composition of the present disclosure comprises chitosan that is 75% or more deacetylated. In still another embodiment, an aerosolized composition of the present disclosure comprises chitosan that is 50% or more deacetylated. The degree of deacetylation (% DD) can be determined by methods commonly known in the art, such as nuclear magnetic resonance (NMR) spectroscopy.

Chitosan is expected to have a positive charge at physiological pH. As polyanionic polysaccharides are negatively charged at that pH, the polyanionic polysaccharides will bind to chitosan by ionic interaction, forming one set of embodiments referred to herein as “chitosan complexes.” As the polyanionic polysaccharides are not expected to bind to all of the charges on the chitosan, it is expected that the chitosan will retain an overall positive charge allowing it to bind to the olfactory epithelium.

In embodiments of chitosan compositions and chitosan complexes, it is expected that, for aerosolized formulations, chitosan will be used having a molecular weight between about 3,800 and about 100,000 Daltons, 3,800 and about 80,000 Daltons, 3,800 and about 70,000 Daltons, 3,800 and about 60,000 Daltons, 3,800 and about 50,000 Daltons, 3,800 and about 40,000 Daltons, 3,800 and 30,000 Daltons, and 3,800 and about 20,000 Daltons, with “about” in this sentence meaning ±5,000 Daltons and each successive mentioned range being more preferred than the one before it.

In embodiments in which the chitosan is present in combination with a polyanionic polysaccharide, sialic acid, or both, the weight ratio of chitosan to polyanionic polysaccharide or sialic acid, or both, in the composition lies within the range about 100:1 to 1:1, particularly about 60:1 to 1:1, with “about” in this sentence meaning ±5. In some of these embodiments, the ratio of chitosan to polysaccharide or sialic acid, or both, is about 10:1 to 1:1; in some of these embodiments, the ratio of about 6:1 to 1:1, with “about” in this sentence meaning ±1. In some of these embodiments, the ratio of chitosan to polysaccharide or sialic acid, or both is about 5:1, about 4:1, about 3:1, about 2.5:1, or about 1.5:1, with “about” in this sentence meaning ±0.5.

It is expected that enzymes in the olfactory mucus will over time degrade the chitosan in the chitosan complexes or compositions applied in the inventive methods. Chitosan complexes or compositions containing a polyanionic polysaccharide, sialic acid, or both, are expected to gradually release the polyanionic polysaccharide, sialic acid, or both, as the chitosan degrades. In some embodiments, the chitosan complexes or compositions may further be combined or co-administered with pharmaceutically acceptable excipients, solvents, or buffers to facilitate administration or formation of particles or droplets of the size desired by the practitioner for administration.

### Polyanionic Polysaccharides

As used herein, the term “polyanionic polysaccharide refers to any polysaccharide or polysaccharide derivative that bears multiple negative charges when in solution and which binds to a pathogen. In preferred forms, the negative charges are due to multiple sulfate groups present on the polysaccharide. In some embodiments, the polyanionic polysaccharide is heparin, dextran sulfate, heparan sulfate, or chondroitin sulfate, with the polysaccharides named first being more preferred than those, if any, named after it.

The polyanionic polysaccharides may be naturally sourced or may be synthesized. Similarly, polyanionic polysaccharides used herein may have structures found in nature, or may have synthetic structures. The structures can be modified heparin structures, such as “glycol-split” heparins (see, e.g., Alekseeva, et al., Anal Bioanal Chem., 2014, 406(1):249-65. doi: 10.1007/s00216-013-7446-4; Ni, et al., Molecules, 2016, 21(11):1602. doi: 10.3390/molecules21111602, or “MST-heparin” (see, e.g., Montgomery et al., Proc Natl Acad Sci, 1992, 89(23):11327-11331. doi.org/10.1073/pnas.89.23.11327). Partly hydrolyzed forms of a polysaccharide can be used provided that the biological activity of binding to a pathogen is maintained. “Pathogen” refers to a bacterium, virus, fungus, prion, or parasite that causes a disease, illness, or syndrome in a mammal In some embodiments, the mammal is a canine. In some embodiments, the mammal is a human.

In some embodiments, when the polyanionic polysaccharide is heparin or heparan sulfate, the heparin or heparan sulfate may possess anti-coagulation activity. In alternative embodiments, when the polysaccharide is heparin or heparan sulfate, the heparin or heparan sulfate may be modified to remove anticoagulation activity. Heparin anticoagulant activity is a result of high-affinity binding to antithrombin and heparins without anticoagulant activity retain a number of biological activities. The heparinase activity of heparin can be reduced by a number of procedures, such as selective desulfation, graded N-acetylation, and glycol splitting (see, e.g,. Naggi et al., J Biol Chem. 2005;280(13):12103-12113) that cause loss of antithrombin-binding affinity. See, Poli et al., Blood, 2014;123(10):1564-73. doi: 10.1182/blood-2013-07-515221. Heparan sulfate and its activity are reviewed in, for example, Turnbull et al., Trends Cell Biol, 2001;11(2):75-82. doi: 10.1016/s0962-8924(00)01897-3 and Fuster and Wang, PROGRESS IN MOLECULAR BIOLOGY AND TRANSLATIONAL SCIENCE, 2010, 93:179-212. Anticoagulant heparan sulfate is reviewed in, for example, Shworak et al., PROGRESS IN MOLECULAR BIOLOGY AND TRANSLATIONAL SCIENCE, 2010, 93:153-178.

Some pathogens are known to bind to heparin or other sulfated polysaccharides, such as heparan sulfate or dextran sulfate. See, e.g., Duensing et al., Infect Immun. 1999, 67(9): 4463-4468. Accordingly, in embodiments in which the chitosan complexes or compositions are being administered to reduce exposure to pathogens, the chitosan is preferably complexed with a polyanionic polysaccharide. One study reports that cellular binding of SARS-CoV-2 is dependent on both the ACE2 receptor and heparan sulfate (see, Clausen et al., Cell, 2020, 183(4):1043-1057.e15. doi: 10.1016/j.cell.2020.09.0330, and it has been reported that, in vitro, heparin binds and causes a conformation change in the SARS-CoV-2 spike protein (Mycroft-West, et al., Thrombosis and Haemostasis, 2020, 120(December 2020):1700-1715. doi:10.1055/s-0040-1721319). Thus, heparan sulfate (either native or modified forms of heparan sulfate that retain the ability to bind the spike protein of SARS-CoV-2) are particularly preferred for use in chitosan complexes or compositions intended to reduce exposure of a subject's olfactory epithelium to SARS-CoV-2 virus or to block access of the virus to olfactory nerves or support structures.

Heparin and other sulfated polyanionic polysaccharides also are known to exhibit anti-inflammatory activities via a variety of mechanisms including neutralization of cationic mediators, inhibition of adhesion molecules, and the inhibition of heparanase, all involved in leukocyte recruitment into tissues. Heparin can inhibit the activation of a range of inflammatory cells, an effect that is due in part to the binding and neutralization of inflammatory mediators and enzymes released during an inflammatory response that would otherwise go on to activate such cells. Without wishing to be bound by theory, it is believed this anti-inflammatory activity of heparin and other sulfated polyanionic polysaccharides may reduce activation of glial cells in the olfactory tissues and thereby decrease any inflammatory response of cells in the olfactory epithelium to environmental factors. Thus, in those embodiments in which the chitosan complex or composition applied according to the inventive methods comprises heparin or another sulfated polyanionic polysaccharide, they are expected to have an effect in reducing pathological conditions not only through their action in capturing and sequestering environmental factors, such as pathogens, that would otherwise reach the brain through the olfactory epithelium, but also by providing heparin or other sulfated polyanionic polysaccharides, which themselves reduce inflammatory response in glial cells in the subject's central nervous system that are implicated in advancing or aggravating neurodegenerative diseases and psychiatric disorders.

Without wishing to be bound by theory, it is believed that the chitosan and the polyanionic polysaccharide (or two or more polyanionic polysaccharides) form a polyelectrolyte complex. In some of these embodiments, the polyanionic polysaccharide is preferably heparin (with or without coagulation or anti-coagulation activity), or dextran sulfate.

### Sialic Acids

Sialic acid are a class of alpha-keto acid sugars with a nine-carbon backbone. According to Wikipedia, the most prevalent member of this group is N-acetylneuraminic acid. In some embodiments, any member of this class of acid sugars can be immobilized on chitosan for use in the inventive methods. In preferred embodiments, the sialic acid or acids immobilized (or “complexed”) on chitosan are sialic acid α-2,3 galactose (“SA α-2,3 gal”), sialic acid α-2,6 galactose (“SA α-2,6 gal”), or both. Methods for conjugating chitosan with sialic acid are known in the art, as exemplified by Dhavale, “Sialic Acid Conjugated Chitosan for the Attenuation of Amyloid-beta Toxicity,” 2009, LSU Master's Theses 3810, digitalcommons.lsu.edu/gradschool_theses/3810, pp. 50-58.

### Administration of the Chitosan Complexes and Compositions

As noted in a preceding section, the anatomy of the nose and the flow characteristics it imparts to air inhaled by the subject allow odor molecules, viruses, and bacteria to access the olfactory epithelium, while keeping out larger particles. Devices such as metered dose inhalers and nebulizers generally do not succeed in applying particulate agents to the olfactory mucosa without being specially configured for this purpose or having the subject engage in specialized breathing patterns. U.S. Pat. No. 7,231,919 (the “'919 patent”) to Kurve Technology, Inc., for example, discloses a nasal adaptor for nebulizers that direct nebulized particles around the curved surfaces of the nose to allow them to reach the olfactory mucus. The claims indicate that the nasal adaptor is able to deliver particles of 2 microns to 50 microns in size. Other delivery systems that are believed to be useable in, or adapted for use in, the inventive methods include Exhalation Delivery Systems “EDS” (Optinose, Yardley, PA), Precision Olfactory Delivery® “POD” (Impel Neuropharma, Seattle, WA, USA), SipNose devices (SipNose, Yokneam, Israel), AeroPump (Aero Pump, Hochheim, Germany), Metered Nasal Dispenser (Pharmasystem, Markham ON, Canada), Mistette MK Pump II, GL18 (MeadWestvaco Calmar, Hemer, Germany), and SP270+ (Nemera, La Verpillière, France).

Chitosan complexes and compositions for use in the inventive methods are preferably delivered by a device configured to provide localized delivery through the olfactory cleft to the olfactory epithelium. The complexes and compositions may be formulated as a liquid solution, suspension, or dry powder and loaded into a suitable dispenser for administration to the OE. Whether as liquid droplets or dry powder, the complexes and compositions are preferably applied to the OE as particles of 5.5 microns in diameter to 30 microns.

As practitioners will appreciate, the olfactory clefts which contain the olfactory epithelium are two narrow vertical passages at the top of the nasal cavities, which have a limited surface area. As the object of the present invention is to cover some or all of the surface of the olfactory epithelium within the clefts with the chitosan complexes and compositions, rather than to have the compositions absorbed or act internally, use of the chitosan complexes and compositions to block access of environment factors to the olfactory epithelium requires only a small amount of chitosan complex or composition. Referring to a formulation in which the chitosan complex or composition of choice is administered in the form of liquid droplets in a non-sterile or, preferably sterile, saline buffer, it is contemplated that administration of 1.0 mL±0.1 mL into each nare, or an equivalent amount of a powder formulation, will serve to coat the OE. In some embodiments, the administration is of 0.75 mL±0.25 mL. In some embodiments, the administration is of 0.50 mL±0.25 mL. In some embodiments, the administration is of 0.25 mL±0.15 mL. In some embodiments, the administration is of 0.25 mL-0.33 mL. It is not anticipated that doses higher than 1 mL per nare will need to be used. If the practitioner wishes, however, to administer a dose higher than 1 mL per nare, for the patient's comfort, it is preferable to divide the dose and administer 1 mL or less at a time, with 10 to 15 minutes between administrations.

Chitosan is preferably present in the chitosan complexes or compositions at a concentration of 6% to 0.01%, 5% to 0.025%, 4.0% to 0.03%, 3.5% to 0.05%, 3.0% to 0.075%, 2.5% to 0.1%, 2% to 0.2%, 1.5% to 0.3%, 1.25% to 0.35%, 1% to .375%, 0.9% to 0.4%, 0.8% to 0.45%, 0.75% to 0.50%, 0.7% to 0.5%, 0.65% to 0.55%, 0.625% to 0.575%, or 0.6% chitosan, and each successively stated concentration being preferred to the one before it. When the chitosan is administered complexed with a polyanionic polysaccharide, such as heparin or a sialic acid, the polysaccharide or sialic acid is preferably present at a concentration of 2% to 0.01%, 1.5% to 0.02%, 1.25% to 0.035%, 1% to .05%, 0.9% to 0.06%, 0.8% to 0.07%, 0.75% to 0.08%, 0.7% to 0.09%, 0.65% to 0.1%, 0.6% to 0.125%, 0.5% to 0.125%, 0.4% to 0.150%, 0.3% to 0.15%, 0.25% to 0.15%, 0.225% to 0.275%, or 0.2%. When the chitosan complex includes both a polyanionic polysaccharide and a sialic acid, each of the two is preferably individually present at the concentrations just mentioned.

It is expected that different formulations of the complexes or compositions will have different residence times during which they will be effective in blocking access of factors to the OE. In some embodiments, the chitosan complex or composition is administered three times a day. In some embodiments, the chitosan complex or composition is administered twice a day. In some embodiments, the chitosan complex or composition is administered daily. In some embodiments, the chitosan complex or composition is administered every two days. In some embodiments, the chitosan complex or composition is administered every three days. In some embodiments, the chitosan complex or composition is administered weekly. In some preferred embodiments, the complexes or compositions are administered for at least two weeks. In some preferred embodiments, the complexes or compositions are administered for at least three weeks. In some preferred embodiments, the complexes or compositions are administered for at least a month.

As previously noted, in embodiments in which the complexes or compositions are being administered to reduce exposure of the subject's brain to environmental factors considered to contribute to the development of Alzheimer's Disease or another neurodegenerative disease or psychiatric disorder, it is contemplated that the chitosan complexes or compositions will be taken on an on-going basis, such as for months or years.

In applications, such as where short-term exposure to a pesticide, an industrial pollutant, or wildfire smoke is expected, the compositions may be administered for a day, a few days, or a week or more, depending on the length of expected exposure. For example, an agricultural worker applying a pesticide to a field for a day will only need to have a composition of chitosan administered for that day, while a park service firefighter fighting a wildfire preferably has a chitosan composition administered on a schedule that maintains protection from smoke particulates for the duration of the time the firefighter is exposed to the smoke. In recent fire seasons in California, for example, firefighters have been on the line for three or more weeks at a time. It is expected that the chitosan complexes and compositions can be used by individual firefighters during these weeks of effort to reduce their uptake through the OE into the brain of particulates in the smoke, including the heavy metals from burning vehicles and structures.

The present disclosure includes the following numbered aspects:
1. A method of blocking access of at least one pathogenic or anthropogenic environmental factor to olfactory epithelium having a proximal side, which proximal side faces an interior surface of a nose of a subject, said method comprising contacting said proximal side of said olfactory epithelium with an effective amount of (a) a chitosan complex, (b) a composition consisting essentially of chitosan (“chitosan composition”), or (c) a mixture of a chitosan complex and a chitosan composition, thereby blocking access of said environmental factors to said olfactory epithelium, provided that said chitosan complex or chitosan composition does not comprise an effective amount of a therapeutic agent to be transported into said brain of said subject.
2. The method of any numbered aspect, wherein said chitosan is 75% or more deacetylated.
3. The method of any numbered aspect, wherein said chitosan complex comprises a polyanionic polysaccharide complexed with said chitosan.
4. The method of any numbered aspect, wherein chitosan and said polyanionic polysaccharide are present in said composition in a ratio from about 100:1 to 10:1.
5. The method of any numbered aspect, wherein said polyanionic polysaccharide is selected from the group consisting of heparin, dextran sulfate, heparan sulfate, and chondroitin sulfate.
6. The method of any numbered aspect, wherein said polyanionic polysaccharide is an anti-coagulant form of heparin.
7. The method of any numbered aspect, wherein said polyanionic polysaccharide is a non-anti-coagulant form of heparin.
8. The method of any numbered aspect, wherein said chitosan complex comprises sialic acid complexed on to said chitosan.
9. The method of any numbered aspect, provided that said chitosan complex consists essentially of chitosan complexed with a polyanionic polysaccharide, chitosan complexed with a sialic acid, or chitosan complexed with both a polyanionic polysaccharide and a sialic acid.
10. The method of any numbered aspect, provided that, if said chitosan complex is provided as a hydrogel, it does not comprise nanoparticles.
11. The method of any numbered aspect, wherein said environmental factor is a pathogen.
12. The method of any numbered aspect, wherein said pathogen is a bacterium, a fungus, a parasite, a prion, or a virus.
13. The method of any numbered aspect, wherein said virus is a coronavirus.
14. The method of any numbered aspect, wherein said coronavirus is SARS-CoV-2.
15. The method of any numbered aspect, wherein said environmental factor is a pesticide.
16. The method of any numbered aspect, wherein said environmental factor is particulate matter.
17. The method of any numbered aspect, wherein said particulate matter is of Particulate Matter (PM)_{2.5} or smaller particles.
18. The method of any numbered aspect, wherein said particulate matter is traffic-related particulates.
19. The method of any numbered aspect, wherein said chitosan complex or said chitosan composition is atomized.
20. The method of any numbered aspect, wherein said chitosan complex or said chitosan composition is applied as a dry powder.
21. The method of any numbered aspect, wherein said chitosan complex or said chitosan composition is applied as a spray.
22. The method of any numbered aspect, wherein said chitosan complex or said chitosan composition is administered via a device configured to apply substances to olfactory epithelium.
23. The method of any numbered aspect, wherein said chitosan complex or said chitosan composition is administered for at least two weeks.
24. The method of any numbered aspect, wherein said chitosan complex or said chitosan composition is administered for at least three weeks.
25. The method of any numbered aspect, wherein said chitosan complex or said chitosan composition is administered for at least one month.
26. The method of any numbered aspect, wherein said contacting is with an effective amount of said chitosan complex.
27. The method of any numbered aspect, wherein said contacting is with an effective amount of said chitosan composition.
28. The method of any numbered aspect, wherein said contacting is with an effective amount of a mixture of said chitosan composition and said chitosan complex.
29. A method of reducing infiltration into the brain of a subject through said subject's olfactory nerves, olfactory support structures, or both, of at least one pathogenic or anthropogenic environmental factor, said method comprising contacting a proximal side of said olfactory nerves, said olfactory support structures, or both, with an effective amount of (a) a chitosan complex, (b) a composition consisting essentially of chitosan (“chitosan composition”), or (c) a mixture of a chitosan complex and a chitosan composition, thereby reducing said infiltration of said at least one environmental factor into said olfactory nerves, said olfactory support structures, or both, provided said composition comprising chitosan does not comprise an effective amount of a therapeutic agent to be transported into said brain of said subject.
30. The method of any numbered aspect, wherein said infiltration of said environmental factors is reduced by at least 5%.
31. The method of any numbered aspect, wherein said infiltration of said environmental factors is reduced by at least 10%.
32. The method of any numbered aspect, wherein said infiltration of said environmental factors is reduced by at least 20%.
33. The method of any numbered aspect, wherein said infiltration of said environmental factors is reduced by at least 25%.
34. The method of any numbered aspect, wherein said chitosan is 75% or more deacetylated.
35. The method of any numbered aspect, wherein said chitosan complex comprises a polyanionic polysaccharide complexed on said chitosan.
36. The method of any numbered aspect, wherein chitosan and said polysaccharide are present in said composition in a ratio from about 100:1 to 10:1.
37. The method of any numbered aspect, wherein said polysaccharide is selected from the group consisting of heparin, dextran sulfate, heparan sulfate, and chondroitin sulfate.
38. The method of any numbered aspect, wherein said polyanionic polysaccharide is a non-anticoagulant form of heparin.
39. The method of any numbered aspect, wherein said polyanionic polysaccharide is an anti-coagulant form of heparin.
40. The method of any numbered aspect, wherein said chitosan complex further comprises sialic acid immobilized to chitosan.
41. The method of any numbered aspect, further provided that said chitosan complex consists essentially of chitosan complexed with a polyanionic polysaccharide, of chitosan complexed with a sialic acid, or of chitosan complexed with both a polyanionic polysaccharide and a sialic acid.
42. The method of any numbered aspect, provided that, if said chitosan complex is provided as a hydrogel, it does not comprise nanoparticles.
43. The method of any numbered aspect, wherein said contacting is with an effective amount of said chitosan complex.
44. The method of any numbered aspect, wherein said contacting is with an effective amount of said chitosan composition.
45. The method of any numbered aspect, wherein said contacting is with an effective amount of a mixture of said chitosan composition and said chitosan complex.
46. The method of any numbered aspect, wherein said environmental factor is a pathogen.
47. The method of any numbered aspect, wherein said pathogen is a bacterium, a fungus, a parasite, a prion, or a virus.
48. The method of any numbered aspect, wherein said virus is a coronavirus.
49. The method of any numbered aspect, wherein said coronavirus is SARS-CoV-2.
50. The method of any numbered aspect, wherein said environmental factor is a pesticide.
51. The method of any numbered aspect, wherein said environmental factor is a particulate mix from an industrial or work operation.
52. The method of any numbered aspect, wherein said chitosan complex, chitosan composition, or mixture of chitosan complex and chitosan composition is atomized
53. The method of any numbered aspect, wherein said chitosan complex, chitosan composition, or mixture of chitosan complex and chitosan composition is applied as a dry powder.
54. The method of any numbered aspect, wherein said chitosan complex, chitosan composition, or mixture of chitosan complex and chitosan composition is applied as a spray.
55. The method of any numbered aspect, wherein said chitosan complex, chitosan composition, or mixture of chitosan complex and chitosan composition is administered via a device configured to apply substances to olfactory epithelium.
56. The method of any numbered aspect, wherein said chitosan complex, chitosan composition, or mixture of chitosan complex and chitosan composition is administered for at least two weeks.
57. The method of any numbered aspect, wherein said chitosan complex is administered for at least one month.
58. A method of reducing infiltration into the brain of a subject through said subject's olfactory nerves, olfactory support structures, or both, of a metal atom or ion, said method comprising contacting a proximal side of said olfactory nerves, said olfactory support structures, or both, with an effective amount of a composition comprising alpha-chitosan complexed with a polyanionic polysaccharide, thereby reducing said infiltration of said metal atom or ion into said olfactory nerves, said olfactory support structures, or both,
   wherein said composition does not comprise an effective amount of a therapeutic agent to be transported into said brain of said subject, and
   wherein said composition has a pH from about 4 to about 6.
59. The method of any numbered aspect, wherein said alpha-chitosan is 75% or more deacetylated.
60. The method of any numbered aspect, wherein said alpha-chitosan and said polyanionic polysaccharide are present in said composition in a ratio from about 100:1 to 10:1.
61. The method of any numbered aspect, wherein said polyanionic polysaccharide is selected from the group consisting of heparin, dextran sulfate, heparan sulfate, and chondroitin sulfate.
62. The method of any numbered aspect, wherein said polyanionic polysaccharide is a non-anticoagulant form of heparin.
63. The method of any numbered aspect, wherein said polyanionic polysaccharide is an anti-coagulant form of heparin.
64. The method of any numbered aspect, wherein said composition further comprises sialic acid.
65. The method of any numbered aspect, wherein the metal atom or ion is Mn.
66. The method of any numbered aspect, wherein said composition is atomized
67. The method of any numbered aspect, wherein said composition is applied as a dry powder.
68. The method of any numbered aspect, wherein said composition is applied as a spray.
69. The method of any numbered aspect, wherein said composition is administered via a device configured to apply substances to olfactory epithelium.
70. The method of any numbered aspect, wherein said composition is administered for at least two weeks.
71. The method of any numbered aspect, wherein said contacting reduces inflammation caused by said metal atom or ion.
72. The method of any numbered aspect, wherein the metal atom or ion is selected from Al, Bi, Cu, Mn, Zn, Ni, Pb, Mg, Fe, Cd, Cu, Hg, and As.
73. A method of reducing infiltration into the brain of a subject through said subject's olfactory nerves, olfactory support structures, or both, of a particulate matter, said method comprising contacting a proximal side of said olfactory nerves, said olfactory support structures, or both, with an effective amount of a composition comprising alpha-chitosan complexed with a polyanionic polysaccharide, thereby reducing said infiltration of said particulate matter into said olfactory nerves, said olfactory support structures, or both,
   wherein said composition does not comprise an effective amount of a therapeutic agent to be transported into said brain of said subject, and
   wherein said composition has a pH from about 4 to about 6.
74. The method of any numbered aspect, wherein said infiltration of said particulate matter is reduced by at least 5%.
75. The method of any numbered aspect, wherein said alpha-chitosan is 75% or more deacetylated.
76. The method of any numbered aspect, wherein said alpha-chitosan and said polyanionic polysaccharide are present in said composition in a ratio from about 100:1 to 10:1.
77. The method of any numbered aspect, wherein said polyanionic polysaccharide is selected from the group consisting of heparin, dextran sulfate, heparan sulfate, and chondroitin sulfate.
78. The method of any numbered aspect, wherein said polyanionic polysaccharide is a non-anticoagulant form of heparin.
79. The method of any numbered aspect, wherein said polyanionic polysaccharide is an anti-coagulant form of heparin.
80. The method of any numbered aspect, wherein said composition further comprises sialic acid.
81. The method of any numbered aspect, wherein said particulate matter is PM_{2.5} or smaller.
82. The method of any numbered aspect, wherein said composition is atomized
83. The method of any numbered aspect, wherein said composition is applied as a dry powder.
84. The method of any numbered aspect, wherein said composition is applied as a spray.
85. The method of any numbered aspect, wherein said composition is administered via a device configured to apply substances to olfactory epithelium.
86. The method of any numbered aspect, wherein said composition is administered for at least two weeks.
87. The method of any numbered aspect, wherein said contacting reduces inflammation caused by said particulate matter.
88. A method of protecting a subject against environmental factors contributing to a neurodegenerative disease and/or a psychiatric disorder, comprising reducing infiltration into the brain of a subject through said subject's olfactory nerves, olfactory support structures, or both, of a metal atom or ion, said method comprising contacting a proximal side of said olfactory nerves, said olfactory support structures, or both, with an effective amount of a composition comprising alpha-chitosan complexed with a polyanionic polysaccharide, thereby reducing said infiltration of said metal atom or ion into said olfactory nerves, said olfactory support structures, or both,
   wherein said composition does not comprise an effective amount of a therapeutic agent to be transported into said brain of said subject, and
   wherein said composition has a pH from about 4 to about 6.
89. The method of aspect 88, wherein the neurodegenerative disease and/or a psychiatric disorder is Alzheimer's disease (AD), Parkinson's disease (PD), schizophrenia, or depression.

### EXAMPLES

### Example 1

This Example sets forth an in vitro uptake assay for testing the ability of any particular chitosan complex or composition of interest, such as a chitosan complexed with a polyanionic polysaccharide such as heparan sulfate, to bind an environmental factor of interest in a solution.
1. Provide an aqueous solution.
2. Add to the solution a known amount of the environmental factor of interest.
3. Add to the solution a selected quantity the chitosan complex or composition to be tested for its ability to bind the environmental factor of interest.
4. Stir the solution to allow the chitosan complex or composition and the environmental factor to come into contact.
5. Measure the amount of the environmental factor remaining in the solution.

A reduction in the amount of the environmental factor remaining in solution compared to the amount added to the solution reflects the ability of the particular chitosan complex or composition tested to bind and neutralize the particular environmental factor it was tested against.

### Example 2

This Example sets forth the results of an in vitro binding assay to determine the ability of chitosan complexes or compositions to bind ⁵²Mn in a solution.

[⁵²Mn]Cl solution in 0.1 M HCl was dried at 90° C. under continuous argon stream, then reconstituted in 0.1 M sodium phosphate, pH 5.5. Following formulation of ⁵²Mn in sodium phosphate, reaction mixtures of ⁵²Mn with chitosan, or heparin-chitosan, were prepared using the original stock concentrations of chitosan and heparin-chitosan provided by the sponsor. Due to addition of ⁵²Mn to aliquots of the stock solution, some dilution of both chitosan and heparin-chitosan occurred. The concentrations of chitosan and heparin-chitosan in the final reaction mixtures were 0.51% chitosan and 0.17%:0.51% heparin-chitosan, respectively.

Three different concentrations of radioactive ⁵²Mn was incubated for 4 hours at 34° C. with compositions of either a) 0.51% alpha-chitosan or b) 0.51% alpha-chitosan and 0.17% heparin at about pH 5.5. Following incubation, the compositions were precipitated by increasing the pH to about 7-7.5 then washed three times with centrifugation between. Unbound ⁵²Mn remained in solution, and an aliquot of the supernatant was collected between each wash. The amount of ⁵²Mn bound to the compositions versus unbound was determined on a dose calibrator via radioactivity determination.

Reaction mixtures were incubated at 34° C. without mixing for four hours in 1.5 mL VWR microcentrifuge tubes. After four hours, the pH was adjusted to 7-7.5 using 40 μL 0.1 M sodium hydroxide at which point the test article precipitation was apparent. The precipitated reaction mixtures were then centrifuged at 18213 rcf for five (5) minutes. An aliquot of the supernatant was transferred to a new microcentrifuge tube and the pellet washed with 100 μL of water and re-centrifuged as above and an aliquot of supernatant removed. This washing was repeated for a total of three water washes. The resultant supernatant and pellets were counted on a dose calibrator for activity determination.

A summary of reaction specifics can be found below for chitosan and heparin-chitosan:

| Specification | Chitosan-1 | Chitosan-2 | Chitosan-3 |
|---|---|---|---|
| ⁵²Mn Activity (μCi) | 87.3 | 46.8 | 20.0 |
| [52Mn] Volume (μL) | 35 | 20 | 8 |
| TA Volume (μL) | 200 | 200 | 200 |
| Total Volume (μL)a | 235 | 235 | 235 |
| Test Article | 0.51% Chitosan | 0.51% Chitosan | 0.51% Chitosan |
| Concentration (%) | | | |
| Activity Concentration | 0.37 | 0.20 | 0.09 |
| (μCi/μL) | | | |

aVolume was made up to 235 μL using 0.1M sodium phosphate buffer, pH 5.5, as needed.

| | Heparin- | Heparin- | Heparin- |
|---|---|---|---|
| Specification | Chitosan-1 | Chitosan-2 | Chitosan-3 |
| ⁵²Mn Activity (μCi) | 85.7 | 46.6 | 19.8 |
| ⁵²Mn] Volume (μL) | 35 | 20 | 8 |
| TA Volume (μL) | 200 | 200 | 200 |
| Total Volume (μL)a | 235 | 235 | 235 |
| Test Article | 0.17% Heparin | 0.17% Heparin | 0.17% Heparin |
| Concentration (%) | 0.51% Chitosan | 0.51% Chitosan | 0.51% Chitosan |
| Activity | 0.36 | 0.20 | 0.08 |
| Concentration | | | |
| (μCi/μL) | | | |

aVolume was made up to 235 μL using 0.1M sodium phosphate buffer, pH 5.5, as needed.

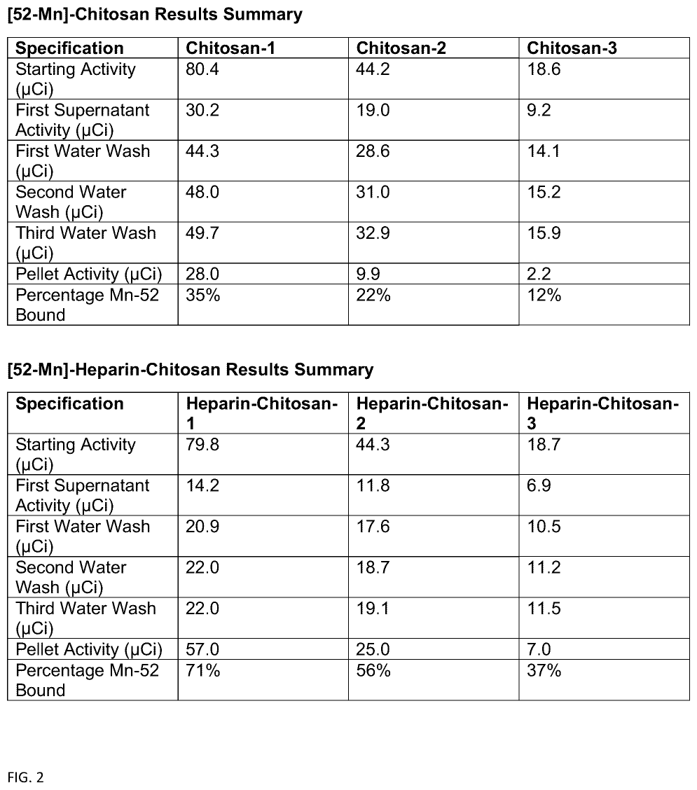

The results of the study are presented in FIG. 2. The tables show that, for all three starting concentrations of ⁵²Mn, compositions comprising alpha-chitosan and heparin bound Mn to a higher degree than compositions comprising alpha-chitosan alone.

### Example 3

This Example sets forth in vitro assays to determine the ability of any particular chitosan complex or composition of interest, such as a chitosan complexed with a polyanionic polysaccharide, to prevent an environmental factor of interest from crossing a barrier.

Assay A. Co-culturing using two-compartment vessels separated by a semipermeable membrane, either coated with the chitosan complex or composition being tested, or left uncoated as a control.

An aqueous solution is disposed in the two compartments. The environmental factor of interest is added to one compartment and the solution in the second compartment is sampled periodically and tested for the presence of the environmental factor of interest to determine whether and how quickly the environmental factor crosses the membrane when the membrane is coated with the chitosan composition being tested.

Assay B. Testing barrier function using a cell monolayer in “electric cell-substrate impedance sensing,” or “ECIS.”

This assay utilizes the fact that cells growing in wells of an ECIS® array (Applied Biophysics, Inc., Troy, NY) having an electrode attach to the electrode and act as insulators, increasing the impedance. When cells are stimulated to change morphology the impedance changes, as the flow of ions is impeded. According to the manufacturer, ECIS® is capable of detecting and quantifying morphology changes in the sub-nanometer to micrometer range. A monolayer of epithelial cells is grown in one or more wells of an ECIS® array. The effect of the chitosan composition of interest on barrier function is measured as the change in impedance over time.

### Example 4

This Example sets forth an exemplary method of making chitosan complexes or compositions.
1. Weight out alpha-chitosan—1.2 g
2. Add 150 ml ultrapure water
3. Add stir bar and adjust pH to 4 using HCl. The solution is stirred for several hours at room temperature. (Some insoluble material will be observed. This is chitin, which will not dissolve.) The solution is stirred until it is somewhat clear with some specks of chitin.
4. The pH is slowly raised to 5 with NaOH. Some chitosan will fall out of solution when adding NaOH. The solution is stirred until the chitosan that has fallen out of solution redissolves.
5. Dissolve heparin (0.4 g for 200 ml) in 50 ml ultrapure water to form a heparin solution.
6. Adding heparin solution to the chitosan solution without vigorous mixing will result in an insoluble precipitate. To get proper complexation, a fast mixer, such as a hand blender, is used. The blender is placed in the chitosan solution and turned on to provide vigorous mixing. The heparin solution is then added to the chitosan solution in a slow stream, making sure that the resulting solution remains under vigorous mixing throughout. After all the heparin solution desired has been added, the vigorous mixing is continued for another minute. The mixing should result in a white solution with the heparin-chitosan in suspension (not perfectly dissolved).

### Example 5

This Example sets forth the results of in vitro studies to determine if treatment of cells with chitosan complexes or compositions reduced the binding to those cells by recombinant SARS-CoV-2 S1/S2 spike protein.

The spike protein of SARS-CoV-2 consists of two non-covalently bound subunits, S1 and S2. See, e.g., Jackson, et al., Nature Rev. Mol. Cell Biol., 2022, 23:3-20. In 2020, a study reported that SARS-CoV-2 spike protein interacts with both cellular heparan sulfate (HS) and angiotensin-converting enzyme 2 (ACE2) through the spike protein's receptor-binding domain, and that HS was an attachment factor that promoted SARS-CoV-2 infection of target cells. See, Clausen, et al., Cell, 2020, 183(4):1043-1057.e15; doi.org/10.1016/j.cell.2020.09.033.

To test whether the inventive chitosan compositions could inhibit binding of SARS-CoV-2 spike protein to susceptible cells, groups of human A549 cells, a cell line of adenocarcinomic human alveolar basal epithelial cells, were (1) left untreated, (2) treated with enzymes to degrade heparan sulfate on the surface of the cells, or (3) treated with heparin/chitosan at different concentrations. The groups of cells were then incubated with recombinant S1 and S2 viral spike protein. Binding of the spike protein to the cells was assessed using a fluorescent antibody and analyzed by flow cytometry.

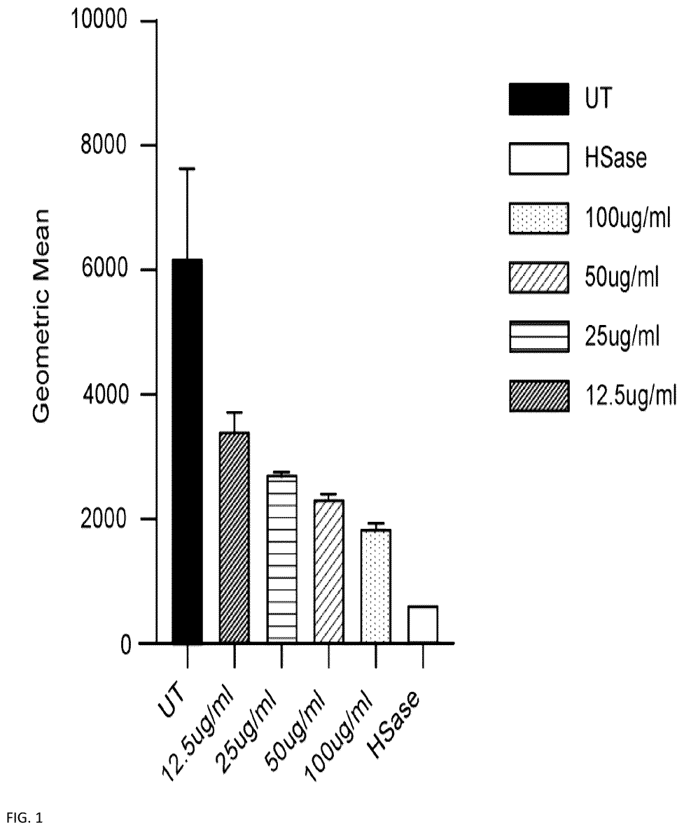

The results of the study are presented in FIG. 1. The graph shows that the heparin-chitosan compositions reduce the binding of spike protein to the cells in a dose dependent manner. As a control, cells were treated with an enzyme (HSase) to degrade heparin sulfate on the cell surface and thereby reduce or remove the ability of HS to serve as an attachment factor for the spike protein.

### Example 6

This Example sets forth an exemplary in vivo assay to determine the ability of any particular chitosan complex or composition of interest, such as a chitosan complexed with a polyanionic polysaccharide, to prevent an environmental factor of interest from entering the brain of a subject in an animal model.

### Uptake of ⁵²Mn in the Brain of Mice or Rats, Through Nose.

Twenty mice or rats (“animals”) are selected and divided into four cohorts of 5.

Cohort 1 (the “test cohort”) has a chitosan complex or composition of interest in a carrier (such as a fluid or a powder) administered daily through the nose to the olfactory epithelium of each animal in the cohort under isoflurane anesthesia. Conveniently, the administration is by spraying the composition onto the olfactory epithelium. Radioactive isotope ⁵²Mn is administered to the animals every other day for a total of three doses, by nasal administration under anesthesia.

The animals in cohort 2 are administered the same chitosan complex or composition as administered to the animals in cohort 1, by the same method, in the same amount and on the same schedule as used for the chitosan complex or composition administered to the animals of cohort 1, but are administered water in place of ⁵²Mn, by the same method, in the same amount, and on the same schedule as used to administer ⁵²Mn to the animals of cohort

The animals in cohort 3 are administered phosphate-buffered saline in place of the chitosan complex or composition administered to the animals of cohort 1, by the same method, in the same amount and on the same schedule as used for the chitosan complex or composition administered to the animals of cohort 1. Saline control or radioactive isotope ⁵²Mn is administered to the animals every other day for a total of three doses, by nasal administration under anesthesia.

The animals of cohort 4 are administered phosphate-buffered saline in place of the chitosan complex or composition administered to the animals of cohort 1, by the same method, in the same amount and on the same schedule as used for the chitosan complex or composition administered to the animals of cohort 1, but are administered water in place of ⁵²Mn, by the same method, in the same amount, and on the same schedule as used to administer ⁵²Mn to the animals of cohort 1.

Any passage of the radioactive Mn through the olfactory epithelium into the brain of a subject animal will result in accumulation in that animal's brain and serum.

In some studies, following administration of the compositions to the respective groups, all the animals are sacrificed and the accumulation of ⁵²Mn in the brains of the animals is measured by brain extraction and gamma counting. In some studies, following administration of the compositions to the respective groups, one animal in each group is sacrificed on each successive day until all the animals have been sacrificed and the accumulation of ⁵²Mn in the brains of the animals is measured brain extraction and gamma counting.

The endpoint analysis is whether of the chitosan complex or composition of interest reduces the amount of ⁵²Mn that accumulates in the brains of the subject animals.

### Example 7

This Example sets forth the results of an in vivo assay to determine the ability of chitosan compositions to prevent the environmental factor Mn from entering the brain of a subject in an animal model.

Twelve rats were selected and divided into three groups of test/control article: 1) 0.2% heparin and 0.6% alpha-chitosan, 2) 0.6% alpha-chitosan, and 3) water. Four rats were placed into each group.

For three days prior to ⁵²Mn administration, each test/control article, at about pH 5.5, was administered via intranasal administration at 50 mL per naris. On day 3, approximately 2 hours post-administration of test/control article, ⁵²Mn was administered to all animals via intranasal installation at the same dose volume. At 4 hours and 24 hours post ⁵²Mn administration, two animals from each group were euthanized at each time point for tissue collection and gamma counting for determination of radioactive content.

At 4 hours and 24 hours post-tracer administration (i.e. ⁵²Mn), n=2 animals from Groups 1-3 were euthanized Following euthanasia, the brain, olfactory bulbs, residual head (post-removal of brain/olfactory bulbs), and remainder of body (residual carcass) were collected, weighed, and gamma counted or placed into a dose calibrator for assessment of radioactive content. The dose calibrator was used when samples were too large to be gamma counted. After completion of radioanalysis for all tissue, the tissues and carcasses were stored for decay and discarded.

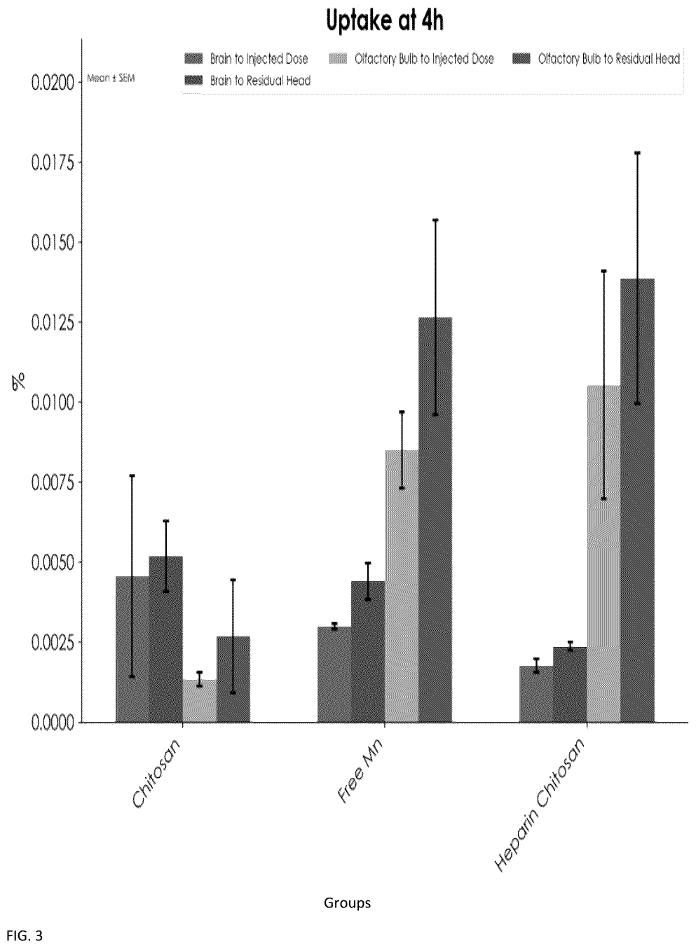

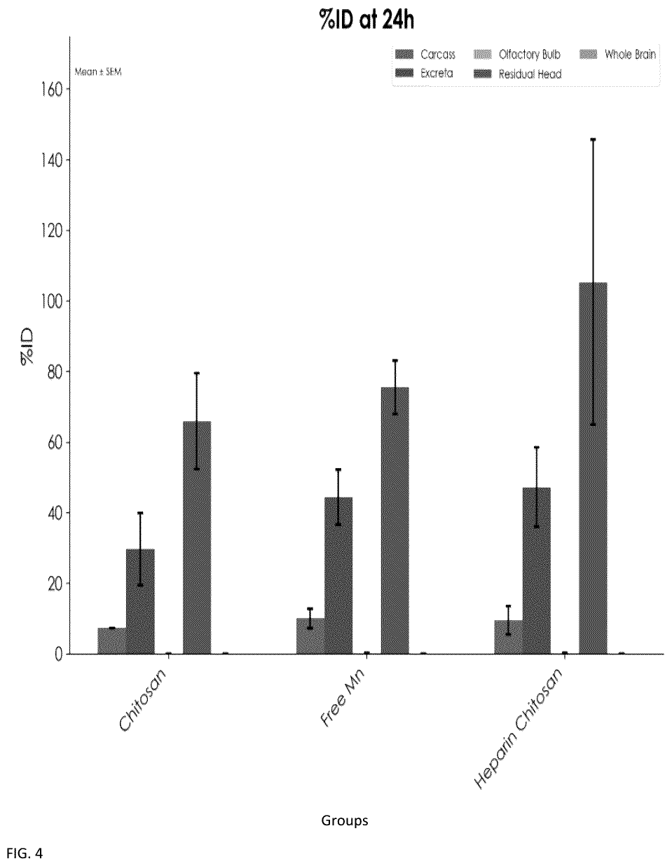

The results of the study are presented in FIGS. 3 and 4. Any passage of the radioactive ⁵²Mn through the olfactory epithelium into the brain results in accumulation in that rat's brain. The graph shows that the group administered chitosan had the highest uptake in brain at 4 hours post-administration, while the group administered a composition comprising chitosan and heparin had the lowest uptake in brain at 4 hours.

For gamma counting, the activity of each collected tissue was measured in units of counts per minute (CPM). Triplicate aliquots of the radiotracer were also assayed in the gamma counter in order to calculate a factor for converting counts to units of activity (μCi/CPM). Values were decay corrected to the time of injection and corrected for background radiation.

For the dose calibrator, the activity of each collected tissue was measured in units of activity (i.e. μCi). Values were decay corrected to the time of injection and corrected for background radiation.

Results were presented in units of percent injected dose (%ID), percent injected dose per gram (%ID/g), and standard uptake values (SUV). The units are calculated the same for ex vivo (gamma counting) results. The definition of these units can be found in the equations below:

The %ID for each analyzed region from the ex vivo gamma counted data can be defined as stated in Equation 1:

%ID=(Uptake/Injected Dose)*100

Where, Uptake=Radioactivity (μCi) in a particular ROI or gamma counting sample, decay-corrected to the time of injection. Injected dose=Radioactivity (μCi) injected into the subject.

The %ID/g for each analyzed region from the ex vivo gamma counted data can be defined as stated in Equation 2:

$$\% \frac{ID}{g} = \frac{\frac{Uptake}{Injected Dose} * 100}{ROI weight}$$

Where, Uptake=Radioactivity (μCi) in a particular ROI or gamma counting sample, decay-corrected to the time of injection. Injected dose=Radioactivity (μCi) injected into the subject. Weight=This is the sample weight of the gamma counted tissue in g. Assumption: tissue density of 1 g/mL.

The Standardized Uptake Value for each analyzed tissue can be defined as stated in Equation 3:

$$SUV = \frac{\frac{Uptake}{Volume}}{\frac{Injected Dose}{Bodyweight}}$$

Where, Uptake=Radioactivity (μCi) in a particular gamma counting sample, decay-corrected to the time of injection. Volume=Sample weight of the gamma counted tissue in g*. Injected Dose=Radioactivity (μCi) injected into the subject. Bodyweight=Subject bodyweight in g. *Assumption: tissue density of 1 g/mL.

Radioactivity measured in excreta (pooled urine and feces) indicated that the complex was significantly more effective at capturing intranasal Mn and facilitating Mn excretion (and preventing its absorption in vivo) than chitosan, alone. Heparin/chitosan@ 47.2% of ingested dose excreted at 24 hours vs. chitosan, only @ 29.7% of ingested dose excreted at 24 hours, reflecting capture of 37% more Mn by 24 hours post-administration.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of reducing infiltration into the brain of a subject through said subject's olfactory nerves, olfactory support structures, or both, of a metal atom or ion, said method comprising contacting a proximal side of said olfactory nerves, said olfactory support structures, or both, with an effective amount of a composition comprising alpha-chitosan complexed with a polyanionic polysaccharide, thereby reducing said infiltration of said metal atom or ion into said olfactory nerves, said olfactory support structures, or both,
wherein said composition does not comprise an effective amount of a therapeutic agent to be transported into said brain of said subject, and
wherein said composition has a pH from about 4 to about 6.

2. The method of claim 1, wherein said alpha-chitosan is 75% or more deacetylated.

3. The method of claim 1, wherein said alpha-chitosan and said polyanionic polysaccharide are present in said composition in a ratio from about 100:1 to 10:1.

4. The method of claim 1, wherein said polyanionic polysaccharide is selected from the group consisting of heparin, dextran sulfate, heparan sulfate, and chondroitin sulfate.

5. The method of claim 1, wherein said polyanionic polysaccharide is a non-anticoagulant form of heparin.

6. The method of claim 1, wherein said polyanionic polysaccharide is an anti-coagulant form of heparin.

7. The method of claim 1, wherein said composition further comprises sialic acid.

8. The method of claim 1, wherein the metal atom or ion is Mn.

9. The method of claim 1, wherein said composition is atomized

10. The method of claim 1, comprising applying the composition as a dry powder.

11. The method of claim 1, comprising applying the composition as a spray.

12. The method of claim 1, comprising applying the composition via a device configured to apply substances to olfactory epithelium.

13. The method of claim 1, comprising applying the composition for at least two weeks.

14. The method of claim 1, wherein said contacting reduces inflammation caused by said metal atom or ion.

15. The method of claim 1, wherein the metal atom or ion is selected from Al, Bi, Cu, Mn, Zn, Ni, Pb, Mg, Fe, Cd, Cu, Hg, and As.

16. A method of reducing infiltration of a particulate matter into a subject's brain through olfactory nerves, olfactory support structures, or both of the subject, said method comprising contacting a proximal side of said olfactory nerves, said olfactory support structures, or both, with an effective amount of a composition comprising alpha-chitosan complexed with a polyanionic polysaccharide, thereby reducing said infiltration of said particulate matter into said olfactory nerves, said olfactory support structures, or both,
wherein said composition does not comprise an effective amount of a therapeutic agent to be transported into said brain of said subject, and
wherein said composition has a pH from about 4 to about 6.

17. The method of claim 16, wherein said infiltration of said particulate matter is reduced by at least 5%.

18. The method of claim 16, wherein said alpha-chitosan is 75% or more deacetylated.

19. The method of claim 16, wherein said alpha-chitosan and said polyanionic polysaccharide are present in said composition in a ratio from about 100:1 to 10:1.

20. The method of claim 16, wherein said polyanionic polysaccharide is selected from the group consisting of heparin, dextran sulfate, heparan sulfate, and chondroitin sulfate.

21. The method of claim 16, wherein said polyanionic polysaccharide is a non-anticoagulant form of heparin.

22. The method of claim 16, wherein said polyanionic polysaccharide is an anti-coagulant form of heparin.

23. The method of claim 16, wherein said composition further comprises sialic acid.

24. The method of claim 16, wherein said particulate matter is PM_{2.5} or smaller.

25. The method of claim 16, wherein said composition is atomized

26. The method of claim 16, comprising applying the composition as a dry powder.

27. The method of claim 16, comprising applying the composition as a spray.

28. The method of claim 16, comprising applying the composition via a device configured to apply substances to olfactory epithelium.

29. The method of claim 16, comprising applying the composition for at least two weeks.

30. The method of claim 16, wherein said contacting reduces inflammation caused by said particulate matter.
